(19) United States
(12) Patent Application Publication
(10) Pub. No.: US 2008/0132962 A1
(43) Pub. Date: Jun. 5, 2008

(54) SYSTEM AND METHOD FOR AFFECTING GATRIC FUNCTIONS

(76) Inventors: Anthony DiUbaldi, Jackson, NJ (US); Michael R. Tracey, Branchburg, NJ (US); Stephen B. Wahlgren, Easton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/565,706

(22) Filed: Dec. 1, 2006

(51) Int. Cl. A61N 1/05

(52) U.S. Cl. 607/2

(57) ABSTRACT

A device for transcutaneous electrical stimulation device for affecting gastric function in a patient and a method for performing the same is provided. The device includes a first waveform generator adapted to generate a first waveform having a first frequency capable of stimulating a vagus nerve of the patient at a predetermined location, a second waveform generator adapted to generate a carrier waveform having a second frequency capable of passing from the surface of skin of the patient at the predetermined location, through tissue to the vagus nerve, a modulation device electrically coupled to the first, second and third waveform generators and adapted to modulate the first and carrier waveforms to create a modulated signal, and a first electrode electrically coupled to the modulation device and positioned substantially adjacent to the skin of the mammal, and adapted to apply the modulated signal thereto.

### BACKGROUND OF THE INVENTION

1. Field of the Invention

The present invention relates generally to devices and methods for selectively stimulating nerves to affect gastric functions, and more particularly to devices and method for surface based stimulation of such nerves.

2. Background Discussion

Obesity has become a major health consideration in much of the developed world. Obesity results from an imbalance between food intake and energy expenditure, which in turn results in a net increase in fat reserves. Excessive food intake, reduced energy expenditure, or both may cause this imbalance.

Appetite and satiety, both of which affect food intake, are partly controlled in the brain by the hypothalamus, which regulates both the sympathetic branch and the parasympathetic branch of the autonomic nervous system. The sympathetic nervous system generally prepares the body for action by increasing heart rate, blood pressure, and metabolism. The parasympathetic system prepares the body for rest by lowering heart rate, lowering pressure, and stimulating digestion. Destruction of the lateral hypothalamus results in hunger suppression, reduced food intake, weight loss, and increased sympathetic activity. In contrast, destruction of the ventromedial nucleus of the hypothalamus results in suppression of satiety, excessive food intake, weight gain, and decreased sympathetic activity. The splanchnic nerves carry sympathetic neurons that supply or innervate the organs of digestion and adrenal glands, and the vagus nerve carries parasympathetic neurons that innervate the digestive system and are involved in the feeding and weight gain response to hypothalamic destruction.

Experimental and observational evidence suggests that there is a reciprocal relationship between food intake and sympathetic nervous system activity. Increased sympathetic activity reduces food intake and reduced sympathetic activity increases food intake. Certain peptides (i.e., neuropeptide Y. galanin) are known to increase food intake while decreasing sympathetic activity. Others such as cholecystokinin, leptin, and enterostatin reduce food intake and increase sympathetic activity. In addition, drugs such as nicotine, ephedrine, caffeine, subitramine, and dexfenfluramine, increase sympathetic activity and reduce food intake.

Efforts to treat obesity include, first and foremost, behavior modification involving reduced food intake and increased exercise. These measures, however, often fail and are supplemented with pharmacological treatments using one or more of the pharmacologic agents mentioned above to reduce appetite and increase energy expenditure. Other pharmacological agents that can cause these affects include dopamine and dopamine analogs, acetylcholine and cholinesterase inhibitors. Pharmacological therapy is typically delivered orally and results in systemic side effects such as tachycardia, sweating and hypertension. In addition, tolerance can develop such that the response to the drug is reduced, even at higher doses.

More radical forms of therapy involve surgery. In general, these procedures reduce the size of the stomach and/or re-route the intestinal system to avoid the stomach. Representative procedures include gastric bypass surgery and gastric banding. These procedures can be very effective, but are highly invasive, require significant lifestyle changes, and can have severe complications.

More recent experimental treatments for obesity involve electrical stimulation of the stomach (gastric electrical stimulation) and the vagus nerve of the parasympathetic system. These therapies use a pulse generator to electrically stimulate the stomach and vagus nerve via one or more implanted electrodes. One such therapy implants electrodes directly onto a bundle of the anterior vagus nerve, near the fundus of the stomach. Electrical signals are transmitted through the electrodes from an attached, implanted pulse generator. The signals are sent at a rate higher than the electrical control activity (ECA) signals that normally occur within the body. The result is distension of the fundus of the stomach and ultimately a sense of fullness. Another known procedure implants the entire system (electrodes and the pulse generator) into the stomach wall.

The intent of any of these therapies is to reduce food intake through the promotion of satiety and/or reduction of appetite. As indicated previously, drug based therapies have many negative side effects, and surgical therapies have obvious disadvantages due to their highly invasive nature. Known electrical based therapies are also invasive in that they require implanted electrodes.

Accordingly, what is needed is an improved and less invasive treatment options for treating obesity.

### SUMMARY OF THE INVENTION

The present invention provides a transcutaneous electrical stimulation device for affecting gastric function in a patient, including a first waveform generator adapted to generate a first waveform having a first frequency capable of stimulating a vagus nerve of the patient at a predetermined location, a second waveform generator adapted to generate a carrier waveform having a second frequency capable of passing from the surface of skin of the patient at the predetermined location, through tissue to the vagus nerve, a modulation device electrically coupled to the first, second and third waveform generators and adapted to modulate the first and carrier waveforms to create a modulated signal, and a first electrode electrically coupled to the modulation device and positioned substantially adjacent to the skin of the mammal, and adapted to apply the modulated signal thereto.

The first and second waveform generators and the electrode may be positioned within a patch device having an adhesive thereon for securing the patch to the skin, and preferred locations for the patch may include the neck region or the lower back region of the patient.

In one embodiment, a return electrode receives the modulated signal, and the first electrode and return electrode are both positioned external of and substantially adjacent to the skin of the mammal, and relative to each other such that the applied modulated signal may pass from the first electrode to the return electrode substantially without passing through tissue of the patient.

In yet another embodiment the first waveform preferably has a frequency of approximately 0.1-40 Hz, and maybe approximately within the range of 0.1-5 Hz. The carrier waveform may preferably have a frequency of approximately 100-400 KHz, and may further be approximately within the range of 170-210 KHz. Further, the first waveform may be a square wave and the carrier waveform may be a sinusoidal wave.

In yet another embodiment, the device further includes a microprocessor adapted to control generation of the first and carrier waveforms by the first and second waveform generators.

The present invention also provides a method for treating obesity in a patient, including generating a first waveform having a frequency capable of stimulating a vagus nerve of the patient, generating a carrier waveform having a frequency capable of passing from the surface of the skin of the patient at a predetermined location, through tissue and to the vagus nerve, modulating the first and carrier waveforms to create a modulated signal, and applying the modulated signal package substantially to the skin surface of the patient at the predetermined location to stimulate the vagus nerve to thereby affect gastric function.

The step of applying the modulated signal may further comprise applying the modulated signal at a frequency sufficiently high to reduce the normal ECA of the patient below approximately 3 beats per minute.

### BRIEF DESCRIPTION OF THE DRAWINGS

FIGS. 1-1b are schematic illustrations of transdermal transmission devices according to selected embodiments of the present invention;

FIGS. 2a and 2b illustrates exemplary waveforms generated by the devices of FIGS. 1 and 1a;

FIG. 3 illustrates one embodiment of a patch within which the present invention may be incorporated;

FIGS. 4a-b illustrate use of the transdermal transmission device in connection with a conductive gel tract;

FIG. 5 illustrates one exemplary placement of the device of FIG. 3; and

FIG. 6 illustrates another exemplary placement of the device of FIG. 3.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. For example, although the present invention is described in detail in relation to stimulation of the vagus nerve and/or muscles in the stomach, the present invention could be used to treat obesity by targeting various other muscles and/or nerves affecting gastrointestinal function.

According to the present invention, a surfaced based or transdermal stimulation system may be used as a gastric electrical stimulation device by stimulating various predetermined body parts involved of the gastrointestinal system, or that otherwise affect the gastrointestinal system. For example, the muscle wall of the stomach and/or the nerves that control “pacing” of the stomach could be appropriate targets. “Pacing” of the stomach refers to the motility of the stomach (i.e., contraction and relaxation of the stomach walls and muscles associated with digestion), which is controlled by electrical signals. Two types of such electrical signals include slow waves, or electrical control activity (ECA) and spike potential, or electrical response activity (ERA). The slow waves serve as a rhythmic pacer, constantly signaling the stomach to pace it at about three “beats” per minute. Spike potentials initiate large contractions of the stomach muscles, which are associated with emptying of the stomach.

The basic sequence of gastric motility involves constant slow wave activity to pace the stomach, and if the stomach remains empty (not distended) the higher level cortex receives no feedback indicative of a sensation of fullness, and the individual will perceive a sense of hunger. Following responsive food intake, the stomach will distend or stretch as it fills. Once this occurs, a signal is sent to the brain signaling fullness via the anterior vagus nerve. Following receipt of this signal the brain sends an ERA signal to the stomach to begin the digestive process, forcing the stomach to contract and empty, and simultaneously secrete digestive juices. As the stomach empties, distension is reduced and the signal indicating fullness ceases. Satiety sensations terminate and the individual again feels hungry.

The surface based stimulation system of the present invention targets muscles and/or nerves involved in the typical sequence of gastric motility to thereby affect sensations of hunger or fullness so as to ultimately affect an obese person's food intake.

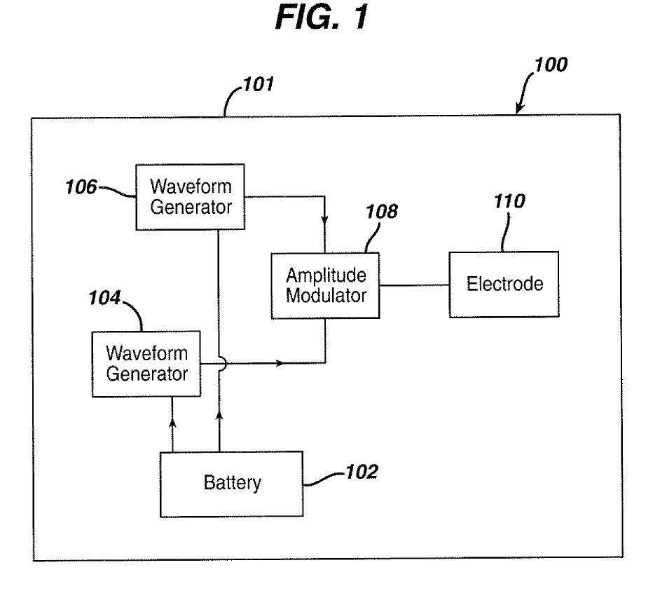

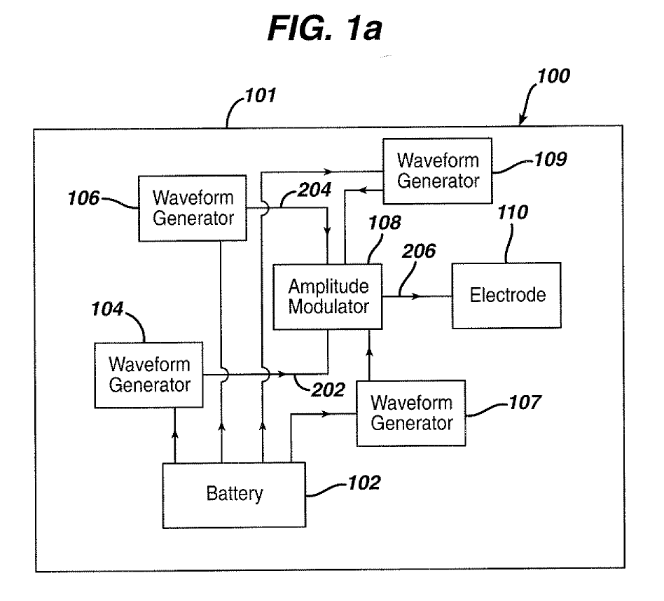

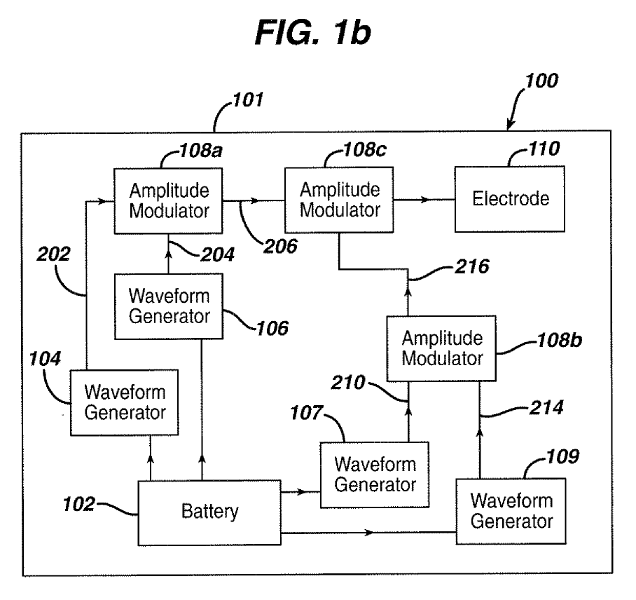

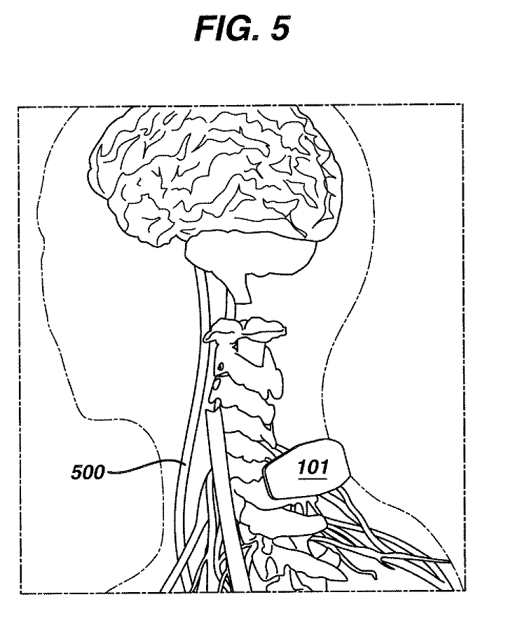

A surface based electrical stimulation device that can be modified for use in the present invention is described in detail in co-pending U.S. application Ser. Nos. 11/146,522, filed on Jun. 7, 2007, Ser. No. 11/343,627, filed on Jan. 31, 2006, and Ser. No. 11/344,285, also filed on Jan. 31, 2006, each of which are incorporated herein by reference in their entirety. As described and illustrated in these previous applications, and as further illustrated in FIGS. 1-4b, an exemplary surface based stimulation device 100 is preferably contained within a patch 101 or the like that can be removably secured to the surface of the skin. For the present application for obesity, a preferred location for the patch is on the left side of the neck (see FIG. 5), so as to target the left vagus.

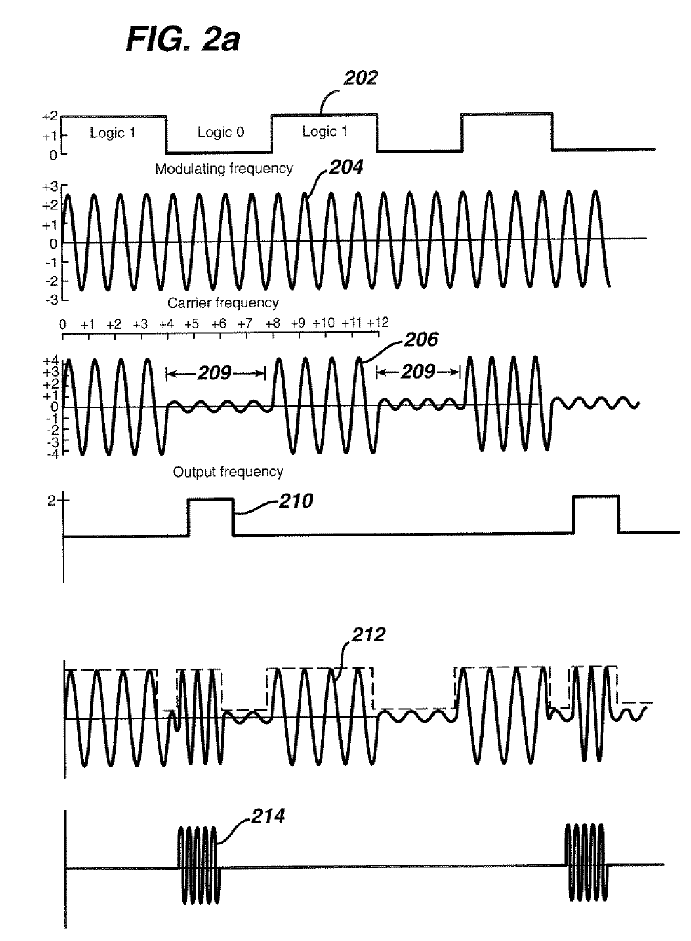

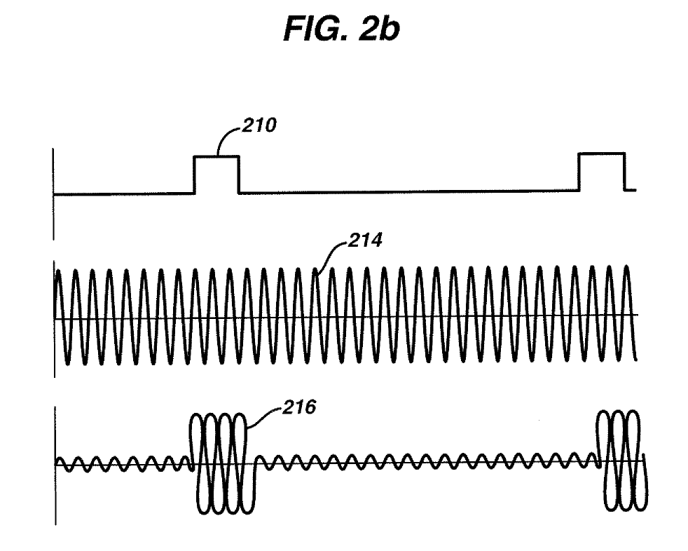

The stimulation or signal transmission device 100 includes a suitable power source 102 such as a lithium ion film battery by CYMBET™ Corp. of Elk River, Minn., model number CPF141490L, and at least first 104 and second 106 waveform generators that are electrically coupled to and powered by the battery. These waveform generators may be of any suitable type, such as those sold by Texas Instruments of Dallas, Tex. under model number NE555. The first waveform generator 104 generates a first waveform 202 (see FIG. 2a) or signal having a frequency known to stimulate a first selected body part, such as the vagus nerve. This nerve is stimulated by a frequency approximately within the range of 0.1-40 Hz, with an optimized frequency preferably being within the range of 0.1-5 Hz. Such a low frequency signal applied to the skin, however, in and of itself, cannot pass through body tissue to reach the targeted vagus nerve with sufficient current density to stimulate the nerve. Thus, the second waveform generator 106 is provided to generate a higher frequency carrier waveform 204, that is applied along with the first waveform to an amplitude modulator 108, such as an On-Semi MC1496 modulator by Texas Instruments. As indicated, the first waveform is preferably a square wave having a frequency of approximately 0.1-40 Hz, and preferably 0.1-5 Hz, and the second carrier waveform is preferably a sinusoidal signal having a frequency in the range of 10-400 KHz, and preferably 170-210 kHz. As those skilled in the art will readily recognize, modulation of this first waveform 202 with the second waveform (carrier waveform) 204 results in a modulated waveform or signal 206 having generally the configuration shown in FIG. 2a. The signals shown in FIGS. 2a and 2b are for illustrative purposes only, and are not intended as true representations of the exemplary signals described herein.

This modulated signal 206 can be provided to an appropriate surface electrode 110, such as DURA-STICK Self Adhesive Electrodes from Chattanooga Group, Inc. of Hixson, Tenn., that applies the modulated waveform directly to the skin. As is readily understood by those skilled in the art, the use of the modulated signal enables transmission of the waveform through tissue due to the high frequency nature of the carrier waveform, yet allows it to be detected (and responded to) by the vagus nerve due to the low frequency envelope of the modulated signal.

Rather than simply applying modulated signal 206 to selectively affect one nerve, the modulated signal 206 has periodic periods of inactivity 209 that can further be taken advantage of to generate a signal package capable of transdermally and selectively stimulating two or more nerves or other body parts if so desired. To accomplish this, a third waveform generator 107 (FIG. 1a) can be used to generate a third waveform having a frequency different from the first waveform and that is specifically selected to stimulate a second nerve or body part. An exemplary third waveform 210 is shown in FIG. 2b. This third waveform must be out of phase with the first waveform 202 to avoid interfering with modulated signal 206. Further, if the frequency ranges that simulate the first and second nerves overlap, the third waveform can be generated or applied during the refractory period of the first nerve to ensure the first nerves inability to respond to this subsequent stimulus. The first 202, second 204 and third 210 waveforms are all applied to amplitude modulator 108, which modulates the three waveforms into a modulated signal package 212. The term “signal package” is used herein to describe a single output signal consisting or three or more individual signals modulated together in any way.

Although one specific embodiment has been described thus far, those skilled in the art will recognize that the appropriate signals may be manipulated in many different ways to achieve suitable modulated signals and/or signal packages. For example, a fourth waveform generator 109 may also be included that generates a fourth carrier waveform 214 having a frequency different from the second carrier waveform. This may be desirable if stimulation of the first and second nerve or body part will require the signal(s) to pass through different types or amounts of tissue. As illustrated, using a single amplitude modulator 108 the fourth carrier waveform 214 must be applied only during periods of inactivity of the first waveform to avoid affecting what would be modulated signal 206. In the alternative, as shown in FIG. 1b, the first waveform 202 and second carrier wave 204 may be provided to a first amplitude modulator 108a to result in a first modulated waveform as shown as 206 in FIG. 2b. Similarly, the third waveform 210 and fourth carrier waveform 214 may be provided to a second amplitude modulator 108b to result in a second modulated waveform 216 as shown in FIG. 2b. These first and second modulated waveforms may be further modulated by a third modulator 108c to create a signal package (i.e., 210) that can be transdermally applied by electrode 110. First and second modulated signals, of course, could also be applied separately via first and second electrodes.

As can be seen from signal package 212, there are still periods of the waveform that are not active. Additional signals can be inserted into these periods to target other frequency independent nerves or other body parts.

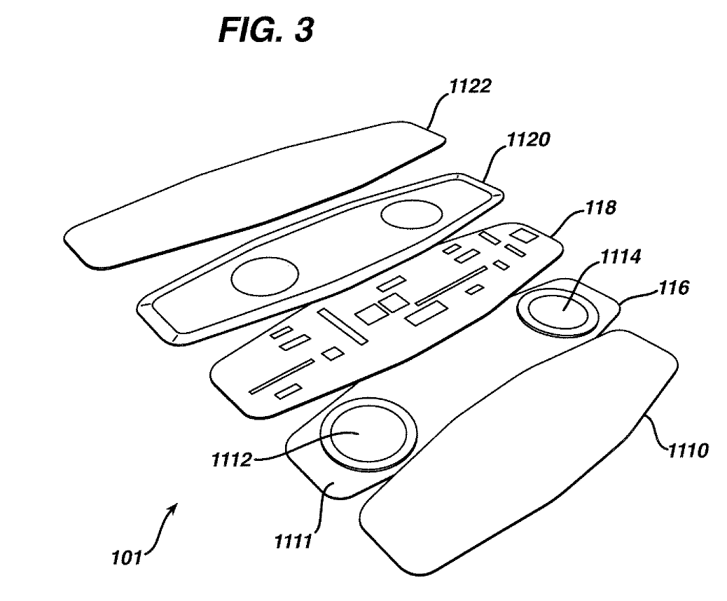

Referring now back to FIG. 3, the transdermal stimulation devices described herein may be incorporated into a transdermal patch 101. This patch may include a first layer 1110 having any suitable adhesive on its underside, with the active and return electrodes 1112, 1114 being secured to the top side 1111 of the first layer. The adhesive layer may further include holes therein (not shown) to accommodate the shape of the electrodes and allow direct contact of the electrodes with the surface of the patient's skin. The electrodes may be secured directly to the first layer, or may be held in place by a second layer 1116 comprised of any suitable material such as a plastic. A third layer 1118 consists of a flexible electronics board or flex board that contains all of the electronic elements described above and that is electrically coupled to the electrodes. A fourth layer 1120 is a thin film battery of any suitable size and shape, and the fifth layer 1122 is any suitable covering such as the plastic coverings commonly used in bandages.

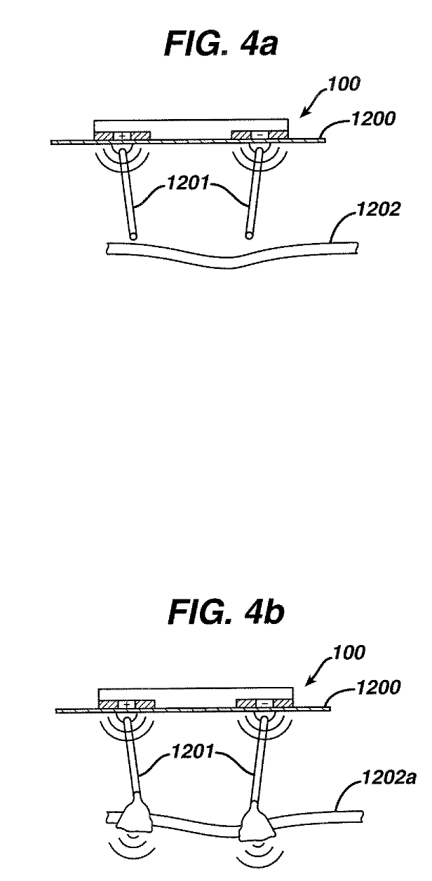

Although capable of being applied transdermally only, the conductance of the stimulation energy from the surface electrode to the target nerve can be increased by the placement of a conductive pathway or “tract” that may extend either fully or partially from the surface electrode to the target nerve as illustrated by FIGS. 4a-4b. The conductive tract may be a cross-linked polyacrylamide gel such as the Aquamid® injectable gel from Contura of Denmark. This bio-inert gel, injected or otherwise inserted, is highly conductive and may or may not be an aqueous solution. The implanted gel provides benefits over rigid implants like wire or steel electrodes. Some of those advantages include ease of delivery, a less invasive nature, and increased patient comfort as the gel is not rigid and can conform to the patient's body. As stated above, the injected gel tract is a highly conductive path from the surface electrode to the target nerve or muscle that will further reduce energy dispersion and increase the efficiency of the energy transfer between the surface electrode and the target nerve or muscle. The conductive gel pathway may provide a conductive pathway from an electrode positioned exterior of the body (i.e., on the skin) or an electrode positioned under the surface of the skin, both of which are considered to be “in proximity” to the skin.

FIG. 4a illustrates an instance where the conductive gel tract 1201 extends from the transdermal stimulation device positioned on the skin 1200 of a patient to a location closer to the targeted muscle, nerve 1202 or nerve bundle. Another advantage of using such a gel material, however, is that unlike rigid conductors (wire), the gel can be pushed into any recessed areas. Wire or needle electrodes can only come in proximity to one plane of the target nerve, whereas the deformable and flowable gel material can envelope, for example, a target nerve 1202a as shown in FIG. 4b. That is, the gel tract can be in electrical and physical contact with the full 360 degrees of the target nerve, thereby eliminating conventional electrode alignment issues. Although described above as extending substantially from the transdermal stimulation device to a position closer to the target nerve, the conductive gel tract could also extend from a location substantially in contact with the target nerve, to a location closer to (but not substantially in contact with) the transdermal stimulation device. Multiple gel pockets or tracts in any configuration could be used.

Although one suitable conductive gel has been described above, various others are also suitable. Many thermoset hydrogels and thermoplastic hydrogels could be used as well. Examples of thermoset hydrogels include cross-linked varieties of polyHEMA and copolymers, N-substituted acrylamides, polyvinylpyrrolidone (PVP), poly(glyceryl methacrylate), poly(ethylene oxide), poly(vinyl alcohol), poly(acrylic acid), poly(methacrylic acid), poly(N,N-dimethylaminopropyl-N′-acrylamide), and combinations thereof with hydrophilic and hydrophobic comonomers, cross-linkers and other modifiers. Examples of thermoplastic hydrogels include acrylic derivatives such as HYPAN, vinyl alcohol derivatives, hydrophilic polyurethanes (HPU) and Styrene/PVP block copolymers.

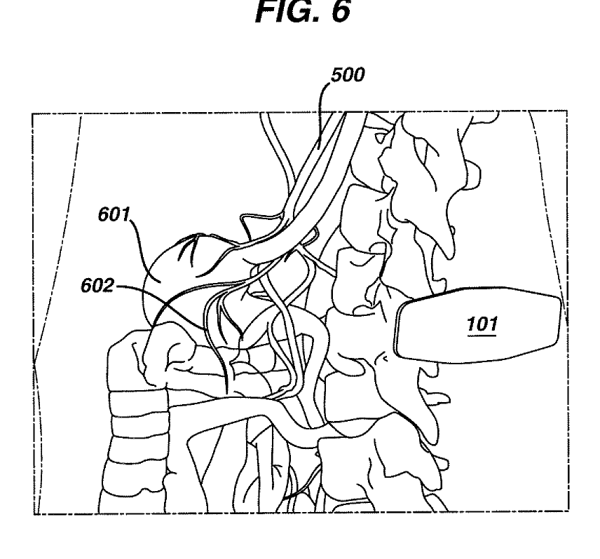

As stated above, a target nerve for use in treating obesity could be the vagus nerve 500. In this instance, a preferred location for placement of the patch 101 would be the back of the neck, and preferably toward the left side as illustrated in FIG. 5. In the alternative, the patch could be placed so as to target the vagus nerve 500 at a location lower down the spine such as in the lower back region where the descending vagus nerve exist the spinal column as shown in FIG. 6. In this location, the patch 101 would preferably be placed over the back in the vicinity of the T5-T9 vertebra.

The above-described transdermal stimulation device 101 can be used to treat obesity by stimulating the vagus nerve to thereby affect the gastric process. As previously indicated, a preferred signal could include a carrier frequency with a frequency greater than or equal to approximately 100-400 kHz (preferably 170-210 kHz) modulated with a lower frequency signal within the range of 0.1-40 Hz (preferably 0.1-5 Hz), having an amplitude of approximately 5 milliamps, and a pulse width of approximately 330 microseconds or greater. The low frequency signal has a frequency higher than signals that are normally sent to the stomach by the vagus nerve that would otherwise result in the normal ERA of approximately 3 beats per minute. This higher frequency has the effect of hyperpolarizing the vagus nerve so as to keep the nerve in the relative and/or refractory period longer than normal so that it fires less frequently than normal. This, in turn, reduces the ERA below 3 beats per minute, causing the patient to feel full and lessening the desire to take in food.

It will be apparent from the foregoing that, while particular forms of the invention have been illustrated and described, various modifications can be made without departing from the spirit and scope of the invention. Accordingly, it is not intended that the invention be limited, except as by the appended claims.

1. A transcutaneous electrical stimulation device for affecting gastric function in a patient, comprising:
a first waveform generator adapted to generate a first waveform having a first frequency capable of stimulating a vagus nerve of the patient at a predetermined location;
a second waveform generator adapted to generate a carrier waveform having a second frequency capable of passing from the surface of skin of the patient at the predetermined location, through tissue to the vagus nerve;
a modulation device electrically coupled to the first, second and third waveform generators and adapted to modulate the first and carrier waveforms to create a modulated signal; and
a first electrode electrically coupled to the modulation device and positioned substantially adjacent to the skin of the mammal, and adapted to apply the modulated signal thereto.

2. The device according to claim 1, wherein the first and second waveform generators and the electrode are positioned within a patch device having an adhesive thereon for securing the patch to the skin.

3. The device according to claim 2, wherein the predetermined location is a neck region or a lower back region of the patient.

4. The device according to claim 1, further comprising a return electrode for receiving the modulated signal, wherein the first electrode and return electrode are both positioned external of and substantially adjacent to the skin of the mammal, and relative to each other such that the applied modulated signal may pass from the first electrode to the return electrode substantially without passing through tissue of the patient.

5. The device according to claim 1, wherein the first waveform has a frequency of approximately 0.1-40 Hz.

6. The device according to claim 5, wherein the first waveform has a frequency of approximately 0.1-5 Hz.

7. The device according to claim 6, wherein the carrier waveform has a frequency of approximately 100-400 KHz.

8. The device according to claim 6, wherein the carrier waveform has a frequency of approximately 170-210 KHz.

9. The device according to claim 8, wherein the first waveform is a square wave and the carrier waveform is a sinusoidal wave.

10. The device according to claim 1, further comprising a microprocessor adapted to control generation of the first and carrier waveforms by the first and second waveform generators.

11. A method for treating obesity in a patient, comprising:
generating a first waveform having a frequency capable of stimulating a vagus nerve of the patient;
generating a carrier waveform having a frequency capable of passing from the surface of the skin of the patient at a predetermined location, through tissue and to the vagus nerve;
modulating the first and carrier waveforms to create a modulated signal; and
applying the modulated signal package substantially to the skin surface of the patient at the predetermined location to stimulate the vagus nerve to thereby affect gastric function.

12. The method according to claim 11, wherein the step of applying the modulated signal further comprises applying the modulated signal at a frequency sufficiently high to reduce the normal ECA of the patient below approximately 3 beats per minute.

13. The method according to claim 11, wherein the frequency of the first waveform is approximately 0.1-40 Hz.

14. The method according to claim 13, wherein the frequency of the first waveform is approximately 0.1-5 Hz.

15. The method according to claim 14, wherein the frequency of the carrier waveform is approximately 100-400 KHz.

16. The method according to claim 15, wherein the frequency of the carrier waveform is approximately 170-210 KHz.

17. The method according to claim 11, wherein the predetermined location is a neck region of the patient or a lower back region of the patient.
